(19) United States
(12) Patent Application Publication
(10) Pub. No.: US 2004/0185481 A1
(43) Pub. Date: Sep. 23, 2004

(54) Testing method using DNA microarray

(75) Inventor: Yasuyuki Numajiri, Kanagawa (JP)

Correspondence Address: FITZPATRICK CELLA HARPER & SCINTO, 30 ROCKEFELLER PLAZA, NEW YORK, NY, 10112, US

(73) Assignee: CANON KABUSHIKI KAISHA, TOKYO (JP)

(21) Appl. No.: 10/765,943

(22) Filed: Jan. 29, 2004

(30) Foreign Application Priority Data
Feb. 6, 2003 (JP) 2003-029666
Feb. 6, 2003 (JP) 2003-029673

(51) Int. Cl. C12Q 1/68; C12M 1/34

(52) U.S. Cl. 435/6; 435/287.2

(57) ABSTRACT

In a testing system, a hybridization pattern is read from a DNA microarray containing a first DNA probe group (region B) which can be used to identify a subject and a second DNA probe group (region A) which can be used to test the health condition of the subject. A pattern corresponding to the first DNA probe group is analyzed from the read hybridization pattern to identify the subject. A pattern corresponding to the second DNA probe group is analyzed from the read hybridization pattern to generate test information.

### FIELD OF THE INVENTION

The present invention relates to a testing technique using a so-called DNA microarray, which tests the health condition by testing genes from a specimen such as blood.

### BACKGROUND OF THE INVENTION

In laboratory tests using blood, saliva, or urine of subjects, a specimen number is added to each container containing a specimen, the correspondence between the specimen numbers and the subjects is recorded, and analysis is then executed by testing apparatuses or the specimens are sent to testing facilities, except cases wherein simple results can be obtained on the spot.

To do medical checkups aiming at preventing diseases or follow up diseases by the laboratory tests, it is important or necessary to compare the results with the past test results. In physical examinations in companies or testing patients in hospitals, subjects are managed by identification numbers (ID numbers). For this reason, it is relatively easy to identify and specify a subject and refer to his/her past test results. However, if a false identification number is input in searching for a subject, the test results of another subject are referred to.

Assume that a person personally undergoes a medical examination in his/her region or sends a specimen for a test by mail or a home delivery service. In a second or subsequent test, the identification number of the subject is often already registered. In this case, when the subject correctly remembers his/her identification number or holds, e.g., a card with the identification number, it is easy to identify and specify the subject and refer to his/her past test results. However, if the subject erroneously remembers his/her identification number or has lost the card with the identification number, it becomes difficult to search for the past test record of the subject.

A name or date of birth is not sufficiently effective in identifying and specifying a subject because there is sometimes a person having the same name or the same date of birth. An address or telephone number can change at times and is therefore not sufficiently reliable in specifying a subject. If the subject is on the spot, his/her fingerprint, voiceprint, iris, or retinal pattern can be used to identify and specify him/her. With only a specimen, however, the subject cannot be identified and specified.

As described above, it is difficult to reliably identify and specify a subject by the conventional method, and past test data cannot be searched for in many cases.

As media to record personal medical information, medical information cards made of an optical card, IC card, or magnetic card are used. Because of the nature of these cards that record medical information, it is impossible or difficult to rewrite data, though a data write is possible. In inputting data to a medical information card, it is very important to confirm whether the holder of the card is an authentic person. This confirmation is done by collating the name, date of birth, photo of face, or fingerprint data recorded on the medical information card. Although voiceprint, iris, and retinal patterns can also be used, they can be confirmed only when the person is on the spot.

However, in laboratory tests of blood, saliva, urine, and stool, data are often input without the presence of the person. Even when the person is present, the correspondence between the specimen and the subject is not always reliable. For this reason, data of another person may erroneously be input to the medical information card. That is, it is difficult to reliably collate the subject of a specimen with the holder of a medical information card by the conventional method. It cannot be denied that data of another person may erroneously be input to the medical information card.

It is recently becoming possible to diagnose cancers of certain types or examine the hepatic function by using a DNA microarray (also called a DNA chip) as one of the laboratory tests. It is expected that tests could be done in the future by using such DNA microarrays to diagnose various diseases or select treatment methods. However, even in tests using DNA microarrays, a number or a bar code is recorded on a DNA microarray itself by engraving and managed. Japanese Patent Laid-Open No. 2001-147231 proposes a technique of mounting an IC memory in a DNA microarray to store and manage the name, sex, number, and the like of a subject. However, neither a method of reliably identifying and specifying a subject corresponding to a DNA microarray nor a method of reliably collating the holder of a medical information card with a subject in inputting test data to the card has been proposed.

### SUMMARY OF THE INVENTION

The present invention has been made in consideration of the above-described problems, and has as its object to make it possible to reliably identify and specify a subject in testing the health condition by using a DNA microarray.

It is another object of the present invention to make it possible to reliably specify a subject in a test using a DNA microarray.

According to one aspect of the present invention, there is provided a testing apparatus using a DNA microarray, comprising: a reading unit configured to read a hybridization pattern in a DNA microarray containing a first DNA probe group which can be used to identify a subject and a second DNA probe group which can be used to test a specimen; an identification unit configured to analyze a pattern corresponding to the first DNA probe group from the hybridization pattern read by the reading unit to identify the subject; and a generation unit configured to analyze a pattern corresponding to the second DNA probe group from the hybridization pattern read by the reading unit to generate test information.

According to another aspect of the present invention, there is provided a testing method using a DNA microarray, comprising: a reading step of reading a hybridization pattern in a DNA microarray containing a first DNA probe group which can be used to identify a subject and a second DNA probe group which can be used to test a specimen; an identification step of analyzing a pattern corresponding to the first DNA probe group from the hybridization pattern read in the reading step to identify the subject; and a generation step of analyzing a pattern corresponding to the second DNA probe group from the hybridization pattern read in the reading step to generate test information.

Furthermore, according to another aspect of the present invention, there is provided a testing apparatus using a DNA microarray, comprising: a reading configured to read a hybridization pattern from a DNA microarray containing a first DNA probe group which can be used to identify a subject; a first acquisition unit configured to analyze a pattern corresponding to the first DNA probe group from the hybridization pattern read by said reading unit to acquire identification information of the subject; a second acquisition unit configured to read a medical information card held by a subject to acquire identification information of the subject recorded on the medical information card; and a comparison unit configured to compare the pieces of identification information acquired by the first and second acquisition unit.

Furthermore, according to another aspect of the present invention, there is provided a DNA microarray comprising: a first DNA probe group which can be used to identify a subject; and a second DNA probe group which can be used to test a health condition of the subject.

Other features and advantages of the present invention will be apparent from the following description taken in conjunction with the accompanying drawings, in which like reference characters designate the same or similar parts throughout the figures thereof.

### BRIEF DESCRIPTION OF THE DRAWINGS

The accompanying drawings, which are incorporated in and constitute a part of the specification, illustrate embodiments of the invention and, together with the description, serve to explain the principles of the invention.

FIG. 1 is a block diagram of a testing system using a DNA microarray according to the first embodiment;

FIG. 2 is a schematic view of a DNA microarray used in the first embodiment;

FIG. 3 is a schematic view of an example of a hybridization pattern;

FIG. 4 is a flow chart for explaining processing procedures in the testing system according to the first embodiment;

FIGS. 5A, 5B, and 5C are views showing the data structures of registration data according to the first embodiment;

FIG. 6 is a block diagram showing the arrangements of a DNA microarray testing system 1 and optical card read/write system 2 according to the second embodiment;

FIG. 7 is a flow chart for explaining processing procedures in the DNA microarray testing system 1 according to the second embodiment;

FIG. 8 is a flow chart for explaining processing procedures in the optical card read/write system 2 according to the second embodiment; and

FIG. 9 is a flow chart for explaining processing procedures in an optical card read/write system 2 according to the third embodiment.

### DETAILED DESCRIPTION OF THE PREFERRED EMBODIMENTS

Preferred embodiments of the present invention will now be described in detail in accordance with the accompanying drawings.

<First Embodiment>

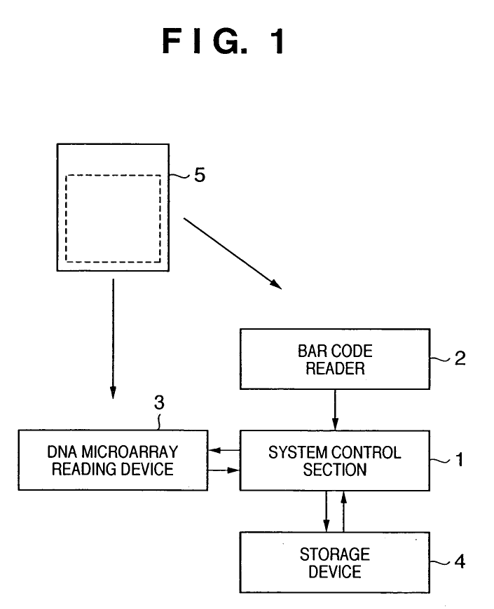

FIG. 1 is a block diagram of a testing system using a DNA microarray according to the first embodiment. The output of a bar code reader 2, the input/output of a DNA microarray reading device 3, and the input/output of a storage device 4 such as a hard disk are connected to a system control section 1 which controls the entire system.

Test processing in the testing system with the above arrangement will be described. First, the following reaction is caused on a DNA microarray 5 in advance. A DNA is extracted, by a DNA extractor, from blood sampled from a subject. The extracted DNA is hybridized with the DNA microarray 5 in a reaction device.

The DNA microarray 5 is formed by arraying several ten to several hundred thousand types of DNA probes on a solid phase surface such as a glass plate having a size of about 1 inch square at a high density. As a characteristic feature, when hybridization reaction with a sample DNA is caused by using the DNA microarray 5, a number of genes can be tested at once. As another characteristic feature, since the DNA probes are periodically arrayed in a matrix, each of them can easily be extracted by using its address (e.g., the position of an intersection between a certain row and a certain column) as information. As genes to be tested, genes of gene polymorphism of individuals are known as well as disease-related genes.

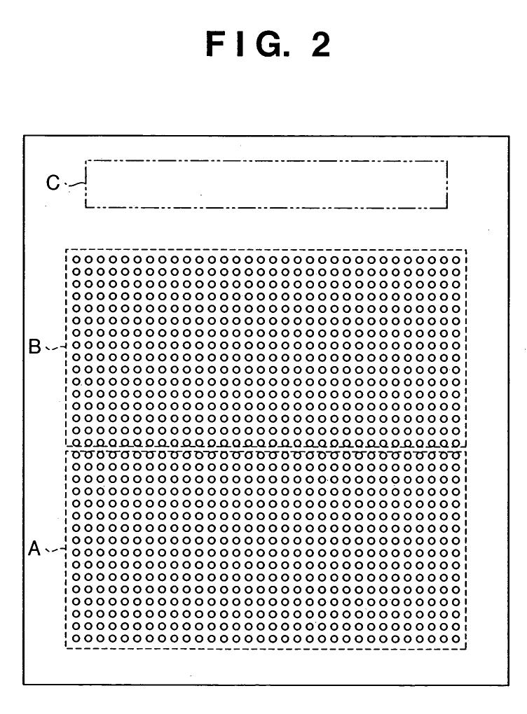

FIG. 2 shows the DNA microarray 5 of the first embodiment. DNA probes with different sequences are combined to regions indicated by ◯ in FIG. 2. A region A shown in FIG. 2 has a DNA probe group which should be used to test the health condition including a disease as the original testing target of the DNA microarray 5. The region A forms a testing DNA probe array portion. The region A may have only an array of DNA probes to test the disease as the original testing target. If there is a sufficient area, DNA probes corresponding to a plurality of diseases may be arrayed, and only DNA probes related to the target disease may be detected at the time of analysis. Alternatively, the plurality of diseases may simultaneously be tested.

A region B shown in FIG. 2 has an array of DNA probes which should be used to identify the subject. In this embodiment, as the identifying DNA probes arrayed in the region B, about 500 different types of DNA probes corresponding to MHC (Major Histocompatibility Complex) genes are used. The subject is identified on the basis of a hybridization pattern image obtained by making the DNA probe group in the region B react with the DNA of the subject.

MHC genes are regions where immune system genes most concentrate in the human genome and have received a great deal of attention because their base sequences have been clarified recently (Nature, Vol. 401, pp. 921-923, 1999). The sequences include genes related to compatibility/incompatibility determination in organ transplantation and bone marrow transfusion. Compatibility/incompatibility determination in organ transplantation and bone marrow transfusion is currently done by tests using leukocytes. However, the tests using leukocytes take a long time and can use only a limited amount of information. For these reasons, it is expected that typing by MHC genes should be the mainstream in the future. MHC (HLA antigens for a human) includes three types of class I antigens, HLA-A, HLA-B, and HLA-C, and three types of class II antigens, HLA-DR, HLA-DQ, and HLA-DP.

Each individual receives each type of antigen from each of his/her parents, i.e., a total of 12 types of antigens, which determines the “type” of the individual. Presently, about 1,000 types of genes are identified as HLA-A, HLA-B, HLA-C, HLA-DR, HLA-DQ, HLA-DP. New MHC genes have still been made clear one after another. The number of MHC genes is expected to further increase. Since only 12 types of genes are selected from 1,000 or more types of genes, the combination of types of a person very rarely matches that of another person. In fact, combinations of types scarcely match in bone marrow transfusion or organ transplantation. It indicates that MHC gene patterns are immensely rich in variety so that they can be regarded as a gene group suitable for personal identification. This gene group never changes with advancing years. In this embodiment, using these facts, personal identification is executed by using DNA probes corresponding to 500 of the above-described 1,000 or more types of genes.

A region C shown in FIG. 2 has a bar code. A bar code representing the type of DNA microarray 5 and a serial number that identifies each DNA microarray is recorded in the region C. The bar code may either directly be printed on the DNA microarray 5 or printed on a paper sheet and bonded to the DNA microarray 5.

In this embodiment, a portion where DNA probes to be used to identify a subject are arrayed is clearly separated from a portion where DNA probes to be used to test the health condition including a disease are arrayed. However, these portions may be mixed when each probe is managed by an address.

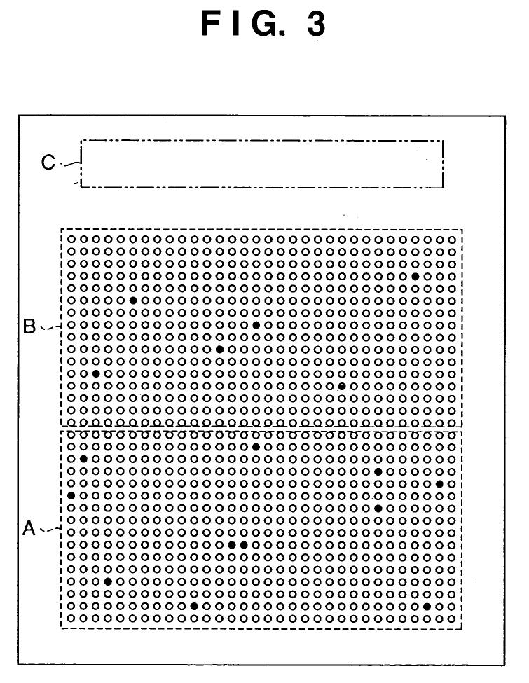

FIG. 3 shows an example of the DNA microarray 5 after the above-described hybridization reaction. Each region indicated by ● represents a probe which forms a hybrid with the DNA of the subject and emits fluorescence. A pattern formed by ● is a hybridization pattern. In the portion (region B) where the DNA probes to be used to identify the subject, a result of hybridization reaction is represented by a binary value, i.e., whether hybridization reaction has occurred or not. That is, there are two types of results representing whether fluorescence is emitted or not (●or ◯). On the other hand, in the portion (region A) having an array of DNA probes to be used to test the health condition including a disease as the original testing target of the DNA microarray 5, an intermediate state (intermediate hybridization reaction) is also present, although it is not illustrated in FIG. 3.

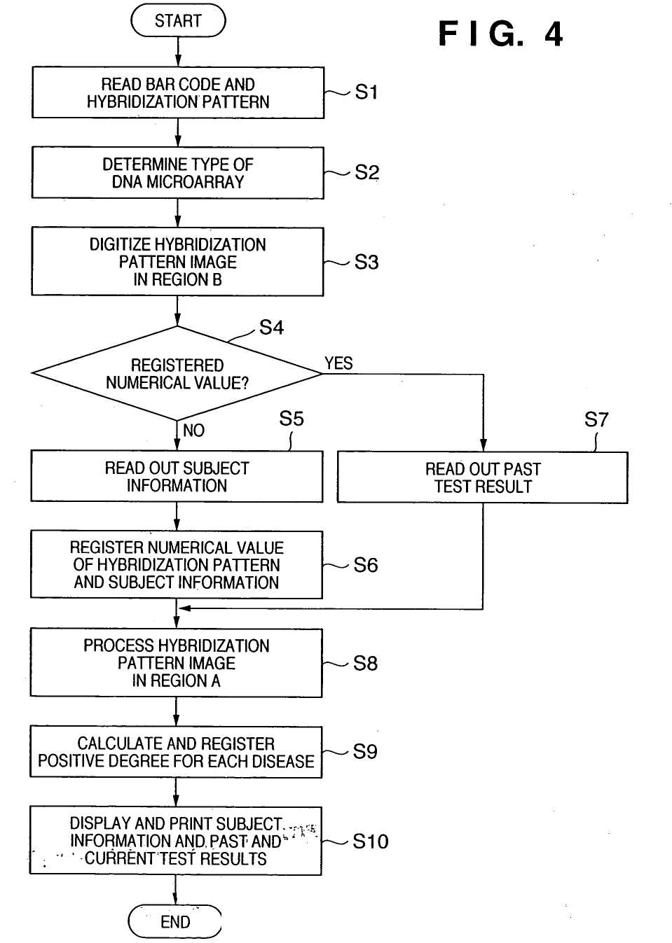

FIG. 4 is a flow chart for explaining processing procedures in the testing system using the DNA microarray 5 according to the first embodiment. In step S1, the system control section 1 of the testing system causes the bar code reader 2 to read the bar code recorded in the region C (FIG. 2) of the DNA microarray 5. The system control section 1 also causes the DNA microarray reading device 3 to read the hybridization pattern.

The bar code in the region C of the DNA microarray 5 is a code that identifies the DNA microarray 5, as described above. In step S2, the system control section 1 determines the type of DNA microarray 5 on the basis of the bar code read result. Accordingly, the disease that is to be tested by using the DNA microarray 5 to be tested can be known. When the DNA microarray 5 whose region B has a different combination of DNA probes to identify the subject is used, its information must also be recorded in the bar code. In this case, an individual is identified on the basis of the type of DNA microarray and the pattern of the region B. In the first embodiment, information is recorded on the DNA microarray 5 by using a bar code, and the information is read by the bar code reader 2. Numbers or characters may be written in place of the bar code and read and recognized by a scanner.

When it is determined that the DNA microarray is, the DNA microarray 5 of this embodiment, a hybridization pattern image for the region B from the DNA microarray reading device 3 is processed to obtain digital data in step S3. As described above, about 500 different types of DNA probes corresponding to MHC genes are arrayed in the region B. Digitization is executed on the basis of which DNA probe on the DNA microarray 5 has hybridized with the DNA of the subject. As described above, since the DNA probes on the DNA microarray 5 are periodically arrayed in a matrix, and each DNA probe can be specified by its address (e.g., the position of an intersection between a certain row and a certain column), digitization is easy. A code obtained by such digitization will be referred to as a personal identification code hereinafter.

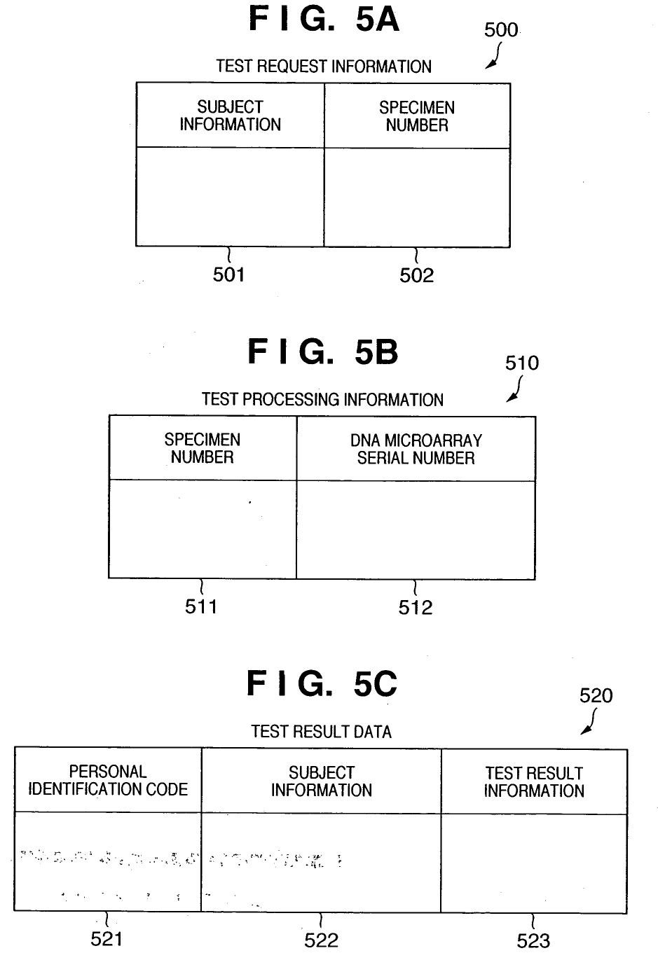

In step S4, the system control section 1 in the testing system compares the personal identification code obtained in step S3 with the numerical values, i.e., personal identification codes of hybridization pattern images already registered in the test result database in the storage device 4. The test result database has a data structure as shown in FIG. 5C, in which a personal identification code 521 obtained by digitizing a hybridization pattern, corresponding subject information 522, and test result information 523 are registered in correspondence with each other. If no code coincides, it is determined that the subject is a new subject, and the flow advances to step S5. In step S5, subject information is acquired from the serial number of the DNA microarray 5, which is contained in the bar code read in step S1 (to be described later with reference to FIGS. 5A and 5B). In step S6, the numerical value (personal identification code 521) corresponding to the hybridization pattern in the region B, which is obtained in step S3, and the subject information 522 (the name, sex, and date of birth of the subject) are registered in the test result database in the storage device 4. Then, the flow advances to step S8.

In the first embodiment, upon receiving a test request, the correspondence between the subject (subject information) and the specimen number is registered in the storage device 4. FIG. 5A shows this registration state. Subject information 501 and a specimen number 502 are registered in test request information 500 in correspondence with each other. In testing, the correspondence between the registered specimen number and the serial number of the DNA microarray 5 to be used for the test is registered in the storage device 4 as test processing information 510. FIG. 5B shows this state. Hence, in step S5, the corresponding specimen number can be specified on the basis of the serial number of the DNA microarray, which is read from the bar code in the region C. The subject information is acquired from this specimen number.

If a numerical value coincides with the personal identification code in step S4, it is determined that the subject has been tested before, and the flow advances to step S7. In step S7, the past test results of the subject are read out from the test result database in the storage device 4. If test results other than those by DNA microarrays are also registered in the test result database, they may also be read out at this time.

Next, the disease as the original testing target of the DNA microarray 5 is tested. In step S8, the hybridization pattern image in the region A (FIG. 3) where the DNA probes for the disease to be tested is arrayed is processed. In step S9, the positive degree for each disease to be tested is calculated and registered in the test result database in the storage device 4. In the first embodiment, the positive degree for each disease is calculated. In accordance with the type of test, information such as the degree of cure of the disease or selection of the treatment method is obtained.

In step S10, the subject information, the current test result, and the past test results, if they are present, are displayed on a monitor (not shown) and/or printed by a printer (not shown). Independently of whether only the subject sees the results or a doctor sees them and makes diagnosis, the past test results are important in prevention or follow-up of a disease.

As described above, the DNA microarray of the first embodiment has the first DNA probe group (region B) which can be used to identify the subject and the second DNA probe group (region A) which can be used to test the health condition of the subject. For this reason, the subject can reliably be identified and specified, and the health condition can be tested.

According to the first embodiment, since the first DNA probe group is formed from probes corresponding to the MHC genes, the probability of identifying and specifying the subject is very high, and stable subject identification can be executed.

According to the first embodiment, there is also a probe identification indicator (region C) to identify the probe structure of at least one of the first and second DNA probe groups. When the probe identification indicator identifies, e.g., the structure of the second DNA probe group, a plurality of different types of second DNA probe groups (regions A) can be used in correspondence with a plurality of types of testing purposes. As a result, necessary tests can reliably be executed.

According to the first embodiment, the DNA microarray contains an identification indicator (serial number) that identifies the DNA microarray itself. When this identification indicator is used, the correspondence to the subject can reliably be confirmed even when, e.g., it is the first test for the subject, and he/she is not registered yet.

According to the testing system of the first embodiment, the hybridization pattern in the DNA microarray (5) containing the first DNA probe group (region B) which can be used to identify the subject and the second DNA probe group (region A) which can be used to test the health condition of the subject is read (DNA microarray reading device 3; S1). A pattern corresponding to the first DNA probe group is analyzed on the basis of the read hybridization pattern to identify the subject (S3). A pattern corresponding to the second DNA probe group is analyzed on the basis of the read hybridization pattern to generate test information (S8 and S9).

As described above, the testing system reads the hybridization pattern on the DNA microarray, identifies the subject by referring to information stored in, e.g., the storage device 4, and tests the necessary health condition. For this reason, the tester can reliably identify the subject and execute the necessary test without any cumbersome operations. The test result can also be stored in the storage device.

The testing system according to the first embodiment stores the subject and past test results (storage device 4) and reads out the past test results of the identified subject. With this arrangement, the health condition considering the change in the past can easily be known. For example, when a person undergoes a test in a hospital, and he/she has already undergone a test using a DNA microarray by the same testing agency in a medical checkup at the company, the subject can easily be identified, and the past test results can be read out. They can be compared with the current result and used for diagnosis.

As described above, according to the testing system of the first embodiment, it can reliably be determined whether the subject has undergone the health condition test using the DNA microarray in the past. If the subject has undergone the test, the past test result can be displayed in addition to the current test result.

The DNA microarray has a first identification indicator (e.g. the bar code in the region C) that indicates the probe structure of the first probe group (region B). The hybridization pattern and the first identification indicator are read (S1; bar code reader 2 and DNA microarray reading device 3). On the basis of the structure of the first DNA probe group recognized on the basis of the first identification indicator, the hybridization pattern of the first DNA probe group is analyzed.

The DNA microarray also has a second identification indicator that indicates the probe structure of the second probe group. The hybridization pattern and the second identification indicator are read. On the basis of the structure of the second DNA probe group recognized on the basis of the second identification indicator, the hybridization pattern of the second DNA probe group is analyzed. The health condition of the subject is tested in accordance with the identification result. Hence, the tester can reliably execute the necessary test without any cumbersome operations.

The DNA microarray has an identification indicator that specifies itself. On the basis of the identification indicator, the subject can be specified.

As described above, according to the first embodiment, in testing the health condition by using the DNA microarray, the subject can reliably be identified and specified.

<Second Embodiment>

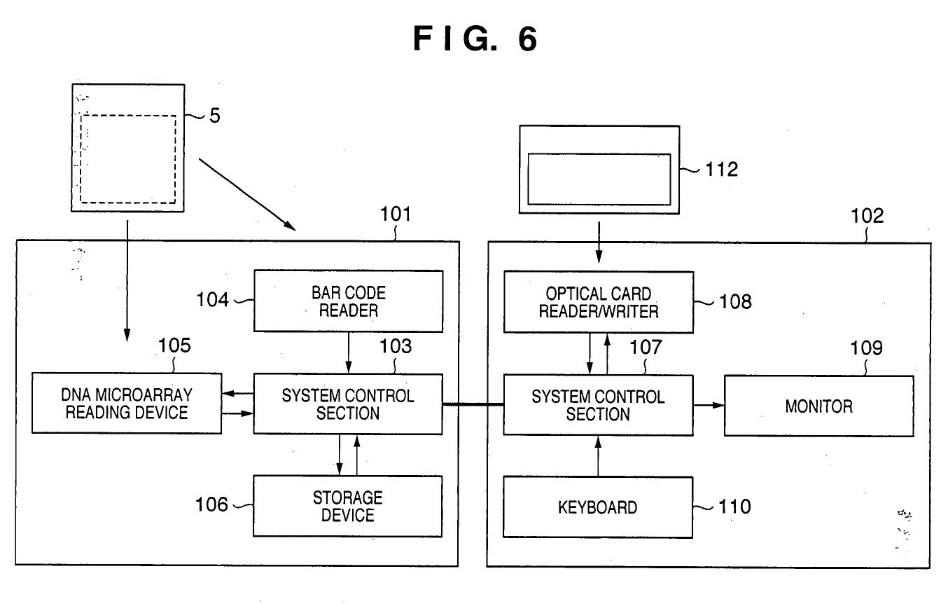

FIG. 6 is a block diagram of a system which writes the test data of a DNA microarray on an optical card in the second embodiment. A DNA microarray test system 101 and optical card read/write system 102 are connected by a network means such as a LAN (Local Area Network).

In the DNA microarray test system 101, a bar code reader 104, DNA microarray reading device 105, and storage device 106 such as a hard disk are connected to a system control section 103 which controls the entire system. In the optical card read/write system 102, an optical card reader/writer 108, monitor 109, and keyboard 110 are connected to a system control section 107 which controls the entire system. In the second embodiment, an optical card 112 is used as a medical information card. Any other portable storage medium such as an IC card or magnetic card, or any other noncard type medium such as an optical disk, magnetic disk, or magnetooptical disk may be used.

A DNA microarray 5 and test processing (hybridization reaction) using it are the same as in the first embodiment, and a description thereof will be omitted here.

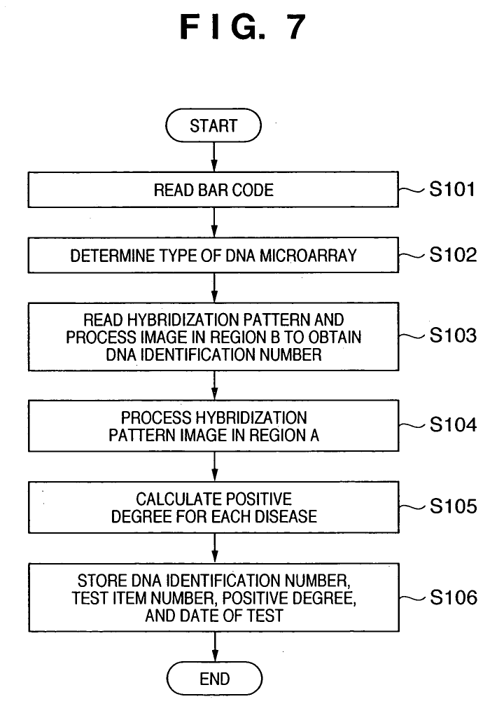

FIG. 7 is a flow chart for explaining processing procedures in the DNA microarray testing system 101 according to the second embodiment. The operator inserts the DNA microarray 5 after hybridization reaction to the bar code reader 104 in advance.

First, in step S101, the system control section 103 of the DNA microarray test system 101 causes the bar code reader 104 to read a bar code recorded in a region C of the DNA microarray 5. A code that identifies the DNA microarray 5 is recorded in this bar code. In step S102, the type of DNA microarray 5 is determined on the basis of part of the bar code. Accordingly, which disease should be tested by the DNA microarray 5 to be tested can be known. The type of test is represented by a test item number.

When the DNA microarray having a different combination of DNA probes (in a region B) to identify the subject is used, its information must also be recorded in the bar code. In this case, the subject is specified on the basis of the hybridization pattern in the region B and the combination type of DNA probes obtained from the bar code. In the second embodiment, information is recorded on the DNA microarray 5 by using a bar code, and the information is read by the bar code reader 104. Numbers or characters may be written in place of the bar code and read and character-recognized by a scanner.

Subsequently, the operator inserts the DNA microarray 5 to the DNA microarray reading device 105. A system that automatically moves the DNA microarray 5 from the bar code reader 104 to the DNA microarray reading device 105 may be used.

When it is determined that the DNA microarray is the DNA microarray 5 of the second embodiment, the hybridization pattern of the DNA microarray is read by the DNA microarray reading device 105 in step S103. Of the hybridization pattern, that for the region B shown in FIG. 3 is processed to obtain digital data, thereby obtaining the DNA identification number of the subject. As described above, about 500 different types of DNA probes corresponding to MHC genes are arrayed in the region B. Since a hybridization pattern unique to each subject is obtained, the subject can be identified.

When the DNA probe on the DNA microarray 5, which has hybridized with the DNA of the subject, can be known, the subject can be specified. As described above, since the DNA probes on the DNA microarray 5 are periodically arrayed in a matrix, and each DNA probe can be specified by its address (e.g., the position of an intersection between a certain row and a certain column), digitization is easy. The digital data can be used to identify the subject.

Next, the disease as the original testing target of the DNA microarray 5 is tested. In step S104, the hybridization pattern image in a region A (FIG. 3) where the DNA probes for the disease to be tested is arrayed is processed. In step S105, the positive degree for each disease to be tested is calculated. In step S106, the DNA identification number obtained in step S103, the test item number read from the bar code, the positive degree obtained in step S105, and the date of test are stored in the storage device 106.

In the second embodiment, the positive degree for each disease is calculated. However, another information may be generated in accordance with the type of test. For example, information such as the degree of cure of the disease or selection of the treatment method may be generated.

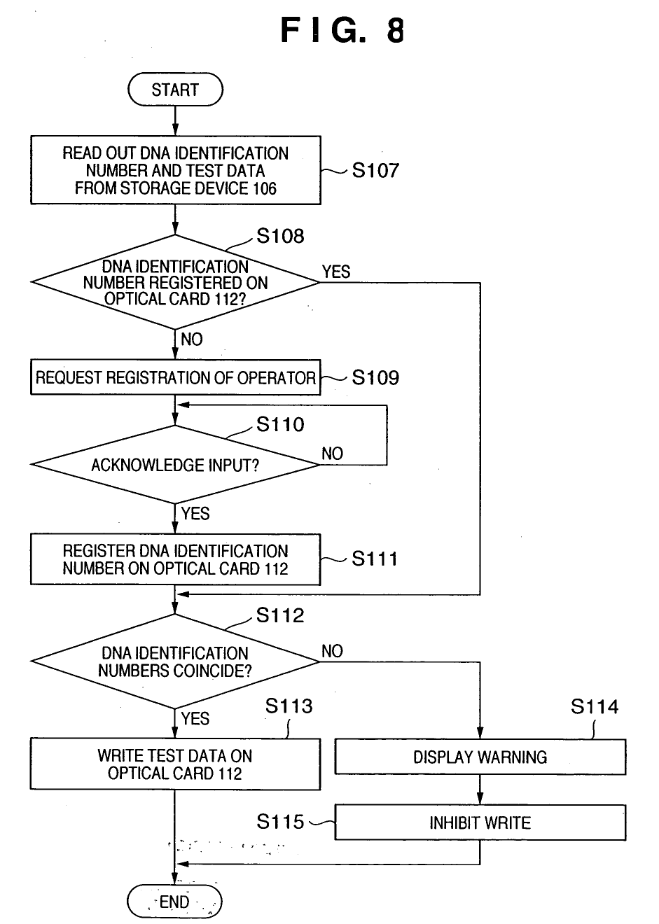

Processing of the optical card read/write system 102 will be described next. FIG. 8 is a flow chart for explaining processing procedures in the optical card read/write system 102 according to the second embodiment. The operator inserts the optical card 112 to the optical card reader/writer 108 in advance.

First, in step S107, the system control section 107 of the optical card read/write system 102 reads out the DNA identification number of the subject and the test item number, positive degree, and date of test as test data from the storage device 106 in the DNA microarray test system 101. In step S108, the optical card reader/writer 108 is controlled to check whether the DNA identification number is registered on the optical card 112. If no DNA identification number is registered, the operator of the optical card read/write system 102 is notified of it in step S109 and prompted to register the DNA identification number. When acknowledgement (for DNA identification number registration) is input by the operator in step S110, the DNA identification number read out in step S107 is registered on the optical card 112 in step S111. If no acknowledgement is input by the operator in step S110, step S110 is repeated after the elapse of a predetermined time to set an acknowledgement input standby state.

When it is determined in step S108 that the DNA identification number is registered on the optical card 112 or after the DNA identification number is registered in step S111, the processing advances to step S112. In step S112, it is determined whether the DNA identification number of the subject from the storage device 106 coincides with that registered on the optical card 112. If YES in step S112, the flow advances to step S113 to write the test result data on the optical card 112. More specifically, the test item number, positive degree, and date of test read out in step S107 are written on the optical card 112

On the other hand, if the DNA identification number read out from the storage device 106 does not coincide with that registered on the optical card 112, the flow advances to step S114 to display a warning on the monitor 109. In step S115, the write of the test data in the storage device 106 is inhibited. In step S114, the operator may be warned by voice instead of displaying the warning.

In the second embodiment, the necessary data are temporarily stored in the storage device 106 and then written on the optical card 112. The test data may directly be written on the optical card 112 without intervening the storage device 106. In the second embodiment, the data on the DNA microarray 5 is read and analyzed first, and then, the data on the optical card 112 are read out. The order may be reversed, or the read may simultaneously be executed.

As described above, according to the second embodiment, the hybridization pattern is read from the DNA microarray (5) containing the first DNA probe group (region B) that can be used to identify the subject. A pattern corresponding to the first DNA probe group is analyzed on the basis of the read hybridization pattern to acquire the identification information (DNA information) of the subject (DNA microarray reading device 105; step S103). In addition, the identification information of the subject is acquired from the medical information card (112) held by the subject (optical card reader/writer 108; step S108). The two pieces of identification information thus acquired are compared to confirm the correspondence between the subject of the DNA microarray and the medical information card (step S112). As a result, the DNA microarray for the health condition test and the medical information card can reliably be collated. The test result from the DNA microarray can reliably be recorded on the medical information card of the correct subject.

According to the second embodiment, the DNA microarray (5) contains the second DNA probe group (region A) that can be used to test the health condition of the subject. A pattern corresponding to the second DNA probe group is analyzed on the basis of the hybridization pattern read from the DNA microarray to generate test information (steps S104 to S106).

According to the second embodiment, when it is determined as the result of comparison of the identification information that the identification information of the subject from the first DNA probe group coincides with the subject identification information recorded on the medical information card, the generated test information is recorded on the medical information card (steps S112 and S113).

Conversely, when the subjects do not coincide as the result of comparison, the write of the test result on the medical information card is inhibited (step S115). Hence, the test result can reliably be prevented from being erroneously written on the medical information card of another person.

According to the second embodiment, when it is determined as the result of comparison of the identification information that the subject identified by the first DNA probe group does not coincide with the subject recorded on the medical information card, a warning is output (step S114). For this reason, the test result can be prevented from being erroneously input to the medical information card of another person.

According to the second embodiment, when the identification information of the subject is not recorded on the medical information card, the identification information acquired in the first acquisition step is recorded on the medical information card (steps S108 to S111). The identification information of the subject can be recorded (registered) on the medical information card without any cumbersome operations by the operator. Hence, the test result from the DNA microarray can correctly be input to the medical information card of the correct subject.

As described above in detail, in the second embodiment, a test result obtained by using a DNA microarray to test the health condition by testing genes from a specimen such as blood can reliably be recorded on a medical information card such as an optical card, IC card, or magnetic card serving as a medium that records personal medical information. More specifically, in a test using a DNA microarray, in inputting the data of the test of the health condition, the subject and the holder of the medical information card can reliably and easily be collated so that the test data can authentically be recorded on the medical information card.

As described above, according to the second embodiment, in the test using the DNA microarray, the subject can reliably be specified.

In the first and second embodiments, about 500 different types of DNA probes corresponding to MHC genes are used. However, the number of DNA probes may be decreased in consideration of other pieces of information to be used to identify the subject. For example, when the name and the date of birth of the subject are used to identify the subject, about 200 different types of DNA probes corresponding to MHC genes may be arrayed on the DNA microarray.

In the first and second embodiments, DNA probes corresponding to MHC genes are used. Genes called SNPs (Single Nucleotide Polymorphism) can also be used as genes suitable for personal identification. These genes may be added in order to add information to the DNA microarray with the MHC genes.

<Third Embodiment>

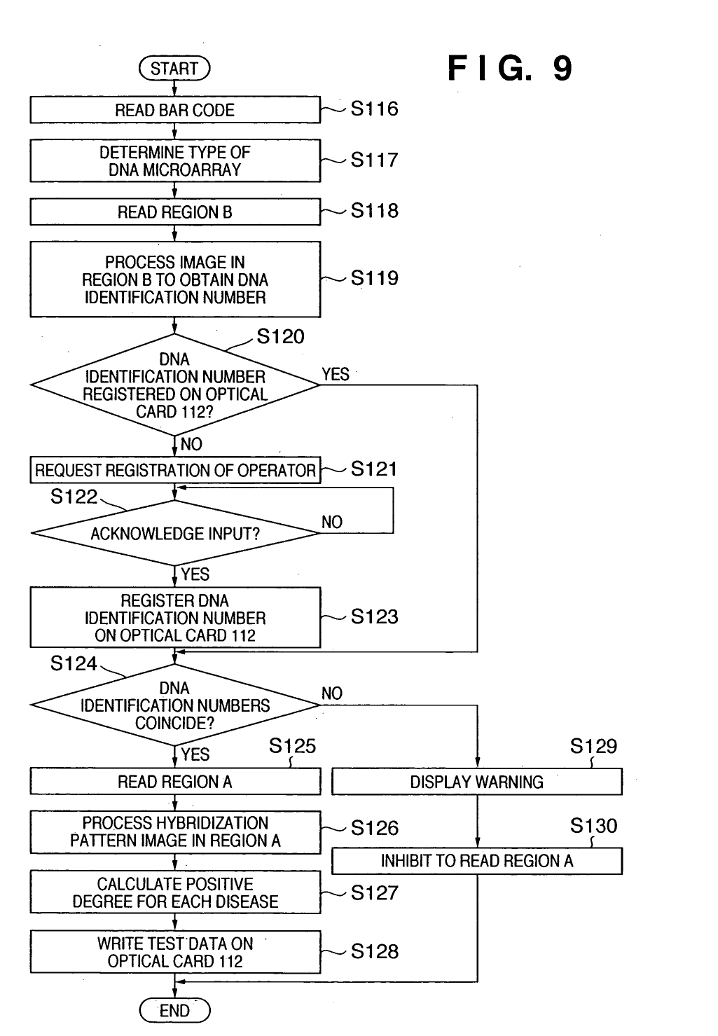

FIG. 9 is a flow chart for explaining processing procedures in an optical card read/write system 102 according to the third embodiment. The entire system configuration including a DNA microarray test system 101 and the optical card read/write system 102 is the same as in the second embodiment. The operator inserts a DNA microarray 5 after hybridization reaction to a bar code reader 104 and an optical card 112 to an optical card reader/writer 108 in advance, as in the second embodiment. In this embodiment, the DNA microarray test system 101 can be controlled from the optical card read/write system 102.

When the operator inputs the start of the test from the optical card read/write system 102, processing starts. As in steps S101 and S102 of the second embodiment, a system control section 103 causes the bar code reader 104 to read a bar code recorded in a region C of the DNA microarray 5 in step S116. In step S117, the type of DNA microarray 5 is determined on the basis of part of the bar code.

Subsequently, the DNA microarray 5 is automatically moved to a DNA microarray reading device 105. In step S118, the hybridization pattern for a region B shown in FIG. 3 is read. In step S119, the hybridization pattern image is processed to obtain digital data, thereby obtaining the DNA identification number of the subject.

As in steps S108 to S111 of the second embodiment, in step S120, the optical card reader/writer 108 is controlled to check whether the DNA identification number is registered on the optical card 112. If no DNA identification number is registered, the operator is notified of it in step S121 and prompted to register the DNA identification number. When the operator confirms the correspondence between the DNA microarray 5, the subject, and the optical card 112 and inputs acknowledgement for DNA identification number registration in step S122, the DNA identification number obtained in step S119 is registered on the optical card 112 in step S123. If no acknowledgement is input by the operator in step S122, step S122 is repeated after the elapse of a predetermined time to set an acknowledgement input standby state.

When it is determined in step S120 that the DNA identification number is registered on the optical card 112 or after the DNA identification number is registered in step S123, the processing advances to step S124. In step S124, it is determined for confirmation whether the DNA identification number of the subject, which is obtained from the DNA microarray 5, coincides with that registered on the optical card 112. If YES in step S124, the flow advances to step S125 to read the hybridization pattern image in the region A (FIG. 3) where DNA probes for the disease to be tested are arrayed. In step S126, the hybridization pattern image is processed. In step S127, the positive degree for each disease to be tested is calculated. In step S128, the test result data is written on the optical card 112.

On the other hand, if the DNA identification number obtained from the DNA microarray 5 does not coincide with that registered on the optical card 112, the flow advances to step S129 to display a warning on a monitor 109. In step S130, the read of the hybridization pattern image in the region A (FIG. 3) where the DNA probes for the disease to be tested are arrayed is inhibited.

According to the third embodiment, when it is determined as the result of comparison of the identification information that the subject identified by the first DNA probe group does not coincide with the subject recorded on the medical information card, the read of the hybridization pattern of the second DNA probe group is inhibited (step S130). For this reason, the test result can reliably be prevented from being erroneously input to the medical information card of another person. In addition, since the test is not executed for any person other than the subject, careless execution of the test can be prevented, and the privacy can be protected.

<Other Embodiments>

Note that the present invention can be applied to an apparatus comprising a single device or to system constituted by a plurality of devices.

Furthermore, the invention can be implemented by supplying a software program, which implements the functions of the foregoing embodiments, directly or indirectly to a system or apparatus, reading the supplied program code with a computer of the system or apparatus, and then executing the program code. In this case, so long as the system or apparatus has the functions of the program, the mode of implementation need not rely upon a program.

Accordingly, since the functions of the present invention are implemented by computer, the program code installed in the computer also implements the present invention. In other words, the claims of the present invention also cover a computer program for the purpose of implementing the functions of the present invention.

In this case, so long as the system or apparatus has the functions of the program, the program may be executed in any form, such as an object code, a program executed by an interpreter, or scrip data supplied to an operating system.

Example of storage media that can be used for supplying the program are a floppy disk, a hard disk, an optical disk, a magneto-optical disk, a CD-ROM, a CD-R, a CD-RW, a magnetic tape, a non-volatile type memory card, a ROM, and a DVD (DVD-ROM and a DVD-R).

As for the method of supplying the program, a client computer can be connected to a website on the Internet using a browser of the client computer, and the computer program of the present invention or an automatically-installable compressed file of the program can be downloaded to a recording medium such as a hard disk. Further, the program of the present invention can be supplied by dividing the program code constituting the program into a plurality of files and downloading the files from different websites. In other words, a WWW (World Wide Web) server that downloads, to multiple users, the program files that implement the functions of the present invention by computer is also covered by the claims of the present invention.

It is also possible to encrypt and store the program of the present invention on a storage medium such as a CD-ROM, distribute the storage medium to users, allow users who meet certain requirements to download decryption key information from a website via the Internet, and allow these users to decrypt the encrypted program by using the key information, whereby the program is installed in the user computer.

Besides the cases where the aforementioned functions according to the embodiments are implemented by executing the read program by computer, an operating system or the like running on the computer may perform all or a part of the actual processing so that the functions of the foregoing embodiments can be implemented by this processing.

Furthermore, after the program read from the storage medium is written to a function expansion board inserted into the computer or to a memory provided in a function expansion unit connected to the computer, a CPU or the like mounted on the function expansion board or function expansion unit performs all or a part of the actual processing so that the functions of the foregoing embodiments can be implemented by this processing.

As many apparently widely different embodiments of the present invention can be made without departing from the spirit and scope thereof, it is to be understood that the invention is not limited to the specific embodiments thereof except as defined in the appended claims.

What is claimed is:

1. A testing apparatus using a DNA microarray, comprising:
a reading unit configured to read a hybridization pattern in a DNA microarray containing a first DNA probe group which can be used to identify a subject and a second DNA probe group which can be used to test a specimen;
an identification unit configured to analyze a pattern corresponding to the first DNA probe group from the hybridization pattern read by said reading unit to identify the subject; and
a generation unit configured to analyze a pattern corresponding to the second DNA probe group from the hybridization pattern read by said reading unit to generate test information.

2. The apparatus according to claim 1, further comprising
a storage unit configured to store the subject and a past test result, and
a unit configured to read out, from said storage unit, the past test result of the subject identified by said identification unit.

3. The apparatus according to claim 1, wherein
the DNA microarray has a first identification indicator which indicates a probe structure of the first DNA probe group,
said reading unit further comprises a unit configured to read the first identification indicator, and
said identification unit analyzes the hybridization pattern of the first DNA probe group read by said reading unit, on the basis of the structure of the first DNA probe group recognized based on the first identification indicator.

4. The apparatus according to claim 1, wherein
the DNA microarray has a second identification indicator which indicates a probe structure of the second DNA probe group,
said reading unit further comprises a unit configured to read the second identification indicator, and
said generation unit analyzes the hybridization pattern of the second DNA probe group read by said reading unit, on the basis of the structure of the second DNA probe group recognized based on the second identification indicator.

5. The apparatus according to claim 1, wherein the DNA microarray has an identification indicator which specifies the DNA microarray itself, and specifies the subject on the basis of the identification indicator.

6. A testing method using a DNA microarray, comprising:
a reading step of reading a hybridization pattern in a DNA microarray containing a first DNA probe group which can be used to identify a subject and a second DNA probe group which can be used to test a specimen;
an identification step of analyzing a pattern corresponding to the first DNA probe group from the hybridization pattern read in the reading step to identify the subject; and
a generation step of analyzing a pattern corresponding to the second DNA probe group from the hybridization pattern read in the reading step to generate test information.

7. The method according to claim 6, further comprising a step of reading out, from a storage unit configured to store the subject and a past test result, the past test result of the subject identified in the identification step.

8. The method according to claim 6, wherein
the DNA microarray has a first identification indicator which indicates a probe structure of the first DNA probe group,
the reading step further comprises a step of reading the first identification indicator, and
in the identification step, the hybridization pattern of the first DNA probe group read in the reading step is analyzed on the basis of the structure of the first DNA probe group recognized based on the first identification indicator.

9. The method according to claim 6, wherein
the DNA microarray has a second identification indicator which indicates a probe structure of the second DNA probe group,
the reading step further comprises a step of reading the second identification indicator, and
in the generation step, the hybridization pattern of the second DNA probe group read in the reading step is analyzed on the basis of the structure of the second DNA probe group recognized based on the second identification indicator.

10. A testing apparatus using a DNA microarray, comprising:
a reading unit configured to read a hybridization pattern from a DNA microarray containing a first DNA probe group which can be used to identify a subject;
a first acquisition unit configured to analyze a pattern corresponding to the first DNA probe group from the hybridization pattern read by said reading unit to acquire identification information of the subject;
a second acquisition unit configured to read a medical information card held by a subject to acquire identification information of the subject recorded on the medical information card; and
a comparison unit configured to compare the pieces of identification information acquired by said first and second acquisition unit.

11. The apparatus according to claim 10, wherein
the DNA microarray contains a second DNA probe group which can be used to test a specimen, and
the apparatus further comprises a generation unit configured to analyze a pattern corresponding to the second DNA probe group from the hybridization pattern read by said reading unit to generate test information.

12. The apparatus according to claim 11, further comprising a first recording unit configured to, when it is determined as a result of comparison by said comparison unit that the subject identified on the basis of the first DNA probe group coincides with that recorded on the medical information card, record the test information generated by said generation unit on the medical information card.

13. The apparatus according to claim 11, further comprising an output unit configured to output a warning when it is determined as a result of comparison by said comparison unit that the subject identified on the basis of the first DNA probe group does not coincide with that recorded on the medical information card.

14. The apparatus according to claim 12, further comprising a second recording unit configured to, when the identification information of the subject is not recorded on the medical information card, record the identification information acquired by said first acquisition unit on the medical information card.

15. A testing method using a DNA microarray, comprising:
a reading step of reading a hybridization pattern from a DNA microarray containing a first DNA probe group which can be used to identify a subject;
a first acquisition step of analyzing a pattern corresponding to the first DNA probe group from the hybridization pattern read in the reading step to acquire identification information of the subject;
a second acquisition step of acquiring identification information of a subject recorded on a medical information card held by the subject; and
a comparison step of comparing the pieces of identification information acquired in the first and second acquisition steps.

16. The method according to claim 15, wherein
the DNA microarray contains a second DNA probe group which can be used to test a specimen, and
the method further comprises a generation step of analyzing a pattern corresponding to the second DNA probe group from the hybridization pattern read in the reading step to generate test information.

17. The method according to claim 16, further comprising a first recording step of, when it is determined as a result of comparison in the comparison step that the subject identified on the basis of the first DNA probe group coincides with that recorded on the medical information card, recording the test information generated in the generation step on the medical information card.

18. The method according to claim 16, further comprising an output step of outputting a warning when it is determined as a result of comparison in the comparison step that the subject identified on the basis of the first DNA probe group does not coincide with that recorded on the medical information card.

19. The method according to claim 17, further comprising a second recording step of, when the identification information of the subject is not recorded on the medical information card, recording the identification information acquired in the first acquisition step on the medical information card.

20. A DNA microarray comprising:
a first DNA probe group which can be used to identify a subject; and
a second DNA probe group which can be used to test a health condition of the subject.

21. The microarray according to claim 20, wherein the first DNA probe group is constituted by probes corresponding to genes of major histocompatibility complex.

22. The microarray according to claim 20, further comprising a probe identification indicator to identify a probe structure of at least one of the first and second DNA probe groups.

23. The microarray according to claim 20, further comprising an identification indicator that identifies the DNA microarray itself.

24. The apparatus according to claim 11, further comprising a reading inhibition unit configured to, when it is determined as a result of comparison by said comparison unit that the identification information of the subject from the first DNA probe group does not coincide with the identification information of the subject recorded on the medical information card, inhibit to read the hybridization pattern of the second DNA probe group.

25. The method according to claim 16, further comprising a reading inhibition step of, when it is determined as a result of comparison in the comparison step that the identification information of the subject from the first DNA probe group does not coincide with the identification information of the subject recorded on the medical information card, inhibiting a read of the hybridization pattern of the second DNA probe group.
